(19) United States
(12) Patent Application Publication
(10) Pub. No.: US 2007/0116672 A1
(43) Pub. Date: May 24, 2007

(54) Treatment of rheumatic diseases

(76) Inventors: Girish J. Kotwal, Louisville, KY (US); Purushottam Jha, Maumelle, AR (US)

Correspondence Address: FISH & RICHARDSON P.C., PO BOX 1022, MINNEAPOLIS, MN, 55440-1022, US

(21) Appl. No.: 10/570,621

(22) PCT Filed: Sep. 3, 2004
(86) PCT No.: PCT/US04/28936

§ 371 Date: Dec. 29, 2006

Related U.S. Application Data
(60) Provisional application No. 60/500,666, filed on Sep. 5, 2003.

(51) Int. Cl. A61K 48/00; A61K 38/16

(52) U.S. Cl. 424/93.2; 514/44; 514/12

(57) ABSTRACT

The present invention relates to the treatment of rheumatic diseases with a vaccinia virus complement control protein (VCP).

### CROSS-REFERENCE TO RELATED APPLICATIONS

This application claims priority under 35 U.S.C. §119(e) of U.S. Application No. 60/500,666, filed Sep. 5, 2003.

### TECHNICAL FIELD

The present invention relates to the treatment of rheumatic diseases with a Poxvirus-encoded complement inhibiting protein such as vaccinia virus complement control protein (VCP).

### BACKGROUND

Rheumatoid arthritis (RA) is a polyarticular inflammatory synovitis that affects up to 1% of the population. The hallmark of RA is progressive destruction of the joints, characterized by synovial hyperplasia, inflammation, and autoimmune phenomena. A variety of mechanisms have been implicated to contribute to the initiation and perpetuation of synovial inflammation, including T-cell activation, persistence and amplification of cytokine networks, and production of pro-inflammatory molecules. Although progress has been made in defining its etiology and pathogenesis, these are still incompletely understood.

Current available therapy for RA is relatively nonspecific and has limited efficacy. For example, both TNF-α receptor molecules and mAbs to TNF-α have been used for treatment of RA. However these treatments have significant side effects. One of the side effects reported is an increased incidence of recurrent tuberculosis in anti-TNF-α-treated individuals. IL-10 and IL-4 are both type 2 cytokines that would be expected to reduce inflammatory responses. However neither has yet been reported to produce significant effects in RA. This may be due to short half-life of cytokines involved. Accordingly, there is a need for methods to treat RA.

### SUMMARY OF THE INVENTION

In one aspect, the invention provides methods for treating a patient having a rheumatic disease or at risk of developing a rheumatic disease. Such methods include administering to the patient an effective amount of Poxvirus-encoded complement inhibiting protein and a pharmaceutically-acceptable carrier. Typically, the effective amount of the Poxvirus-encoded complement inhibiting protein treats at least one symptom associated with the rheumatic disease. As used herein, “treating” refers to ameliorating at least one symptom of a rheumatic disease, or curing and/or preventing the development of a rheumatic disease or condition.

In an embodiment, the rheumatic disease is osteoarthritis, rheumatoid arthritis, juvenile rheumatoid arthritis, fibromyalgia, systemic lupus, erythematosus, scleroderma, a spondyloarthropathy, ankylosing spondylitis, reiter's syndrome, gout, infectious arthritis, lyme disease, polymyalgia rheumatica, polymyositis, psoriatic arthritis, bursitis, or tendinitis. In one embodiment, the rheumatic disease is arthritis (e.g., rheumatic arthritis).

In certain embodiments, administration of the Poxvirus-encoded complement inhibiting protein suppresses inflammation (e.g., joint inflammation), reduces cartilage damage (e.g., cartilage damage in a joint), reduces bone damage or loss (e.g., bone damage or loss in a joint), reduces rheumatoid nodules, inhibits osteoclast formation, and/or prevents or delays the onset of at least one symptom of the rheumatic disease. Representative joints include knees, wrists, fingers, necks, shoulders, elbows, hips, ankles, or foot joints.

In some embodiments, the Poxvirus-encoded complement inhibiting protein is VCP, IMP, smallpox complement inhibiting protein, monkeypox complement inhibiting protein, variola virus complement inhibiting protein. A Poxvirus-encoded complement inhibiting protein can be administered intraperitoneally. A Poxvirus-encoded complement inhibiting protein can be administered into a joint area. A Poxvirus-encoded complement inhibiting protein can be administered in multiple administrations, and can be administered at a dosage of at least about 25 mg/kg (e.g., 25 mg/kg-1000 mg/kg).

According to the invention, a Poxvirus-encoded complement inhibiting protein can be administered in combination with at least one additional agent. Representative agents include nonsteroidal anti-inflammatory drug (NSAID) (e.g., plain aspirin, buffered aspirin, ibuprofen, ketoprofen, naproxen, celecoxib, and rofecoxib), a disease-modifying antirheumatic drug (DMARD) (e.g., an antimalarial agent (e.g., hydroxychloroquine), an immunosuppressant (e.g., methotrexate, azathioprine, cyclosporine, or lefluomide), penicillamine, sulfasalazine, and gold), or a corticosteroid (e.g., prednisone or methylprednisone).

In another aspect, the invention provides for methods of treating a patient having a rheumatic disease or at risk of developing a rheumatic disease. Such a method includes administering to the patient an effective amount of Poxvirus-encoded complement inhibiting protein and a pharmaceutically-acceptable carrier, wherein the Poxvirus-encoded complement inhibiting protein is selected from the group consisting of VCP, IMP, monkeypox complement inhibiting protein, smallpox complement inhibiting protein, and variola virus complement inhibiting protein. Typically, the effective amount of the Poxvirus-encoded complement inhibiting protein treats at least one symptom associated with the rheumatic disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE FIGURES

FIG. 1 depicts a graph showing that VCP suppresses the inflammation in collagen-induced arthritis. Mice were treated with VCP 25 mg/kg i.p. or vehicle (CIA control) alone starting on the 12^{th} day of collagen injection, and paws were observed for clinical arthritic score from day 12 onwards every third day. Values are the mean±SEM of 7 mice/group.

FIG. 2 depicts a graph showing a schedule-dependent efficacy of VCP on arthritic score. Study groups (n=5) were treated with rVCP. In all the treatment regimens, rVCP inhibits the arthritic severity.

FIG. 3 depicts a graph showing the contribution that heparin-binding activity has on the therapeutic effects of VCP. Mice were injected with C-II emulsified in CFA. On day 21 mice with clinical signs of arthritis were randomly divided in three groups. First group (control) received PBS. The second group received truncated rVCP (rVCP-2,3,4) and the third group received full rVCP (rVCP 1,2,3,4). VCP-2,3,4 treatment did not inhibit the clinical course of arthritis.

FIG. 4 depicts a graph showing that VCP treatment reduces the anticollagen antibodies in artic mice. Serum samples were collected from the study groups. Serum anti-CII antibody concentrations were determined by ELISA and expressed as means±SEM (n=5/group). *, p<0.05 rVCP vs. PBS (Student's t test).

FIG. 5 depicts a graph showing that VCP treatment causes reduction in inflammatory cytokine levelsin CIA. Cytokine concentrations in serum samples, collected from the study groups, were assayed by ELISA. Control (grey), PBS treated arthritic mice (black), rVCP treated arthritic mice (white). Data are means±SEM (n=5/group). *, p<0.05 rVCP vs. PBS (Student's t test).

FIG. 6 is a sequence alignment including termini of rVCP constructs and putative heparin binding sites. Multiple alignment of the four short consensus repeats (SCR) from the following orthopoxviruses: vaccinia virus, Copenhagen strain (VAC-COP) (Goebel et al., 1990, Virology, 179: 247-263), vaccinia virus, western reserve strain (VAC-WR) (Kotwal et al., 1989, Virology, 171: 579-587), cowpox virus, Russian isolate from human patient (CPV-GRI) (Schelkunov et al., 1998, Virology, 243:432-460), cowpox virus, Brighton strain (CPV-BRI) (Miller et al., 1995, Cell Immunol., 162:326-332), variola virus, Bangladesh strain (VAR-BSH) (Massung et al., 1994, Virology, 201:215-240), variola major virus, Indian strain (VAR-IND) (Schelkunov et al., 1998, Virology, 243:432-460) variola minor virus, alastrim Garcia strain (VAR-GAR) (Massung et al., 1996, Virology, 221:291-300), and monkeypox virus, isolated from a human patient from Zaire in 1996 (MPV-ZAI). The putative heparin binding sites (K/R-X-K/R) are marked with solid bars; arrows indicated the termini rVCP constructs; and the cysteines are highlighted.

FIG. 7 depicts a structure-function summary table of VCP, VCP homologues, and rVCPs. VCP/IMP/SPICE, MPV homologue of VCP, recombinant VCP, and four recombinant segments of VCP are shown above, along with whether they are able to inhibit hemolysis of sensitized sheep red blood cells and/or bind heparin (IMP=inflammation modulatory protein). Also listed are the number of positively charged amino acids (K+R) found in the protein, percentage of positively charged amino acids (% K+R) making up the protein, pI of the protein, and number of putative heparin binding sites found on the surface of the protein.

### DETAILED DESCRIPTION OF THE INVENTION

Rheumatoid Arthritis (RA)

RA is characterized by infiltration by T cells, B cells, macrophages, and neutrophils into the periarticular spaces and synovial lining. The antigen specific T cells and B cells in the arthritic synovium are believed to be important in initiating the pathogenesis of RA (Initiator Phase). Subsequently, macrophages, neutrophils and lymphocytes are recruited to the joints where they mediate the chronic destructive events (Effector Phase), ultimately resulting in the destruction of neighboring cartilage and bone. Monocytes, macrophages, and synovial fibroblast on stimulation produce cytokines like TNF-α, interleukin-1 (IL-1), and IL-6 as well as soluble mediators of inflammation such as IL-17 and IFN-γ. These infiltrating cells also play an indirect role by stimulating the resident fibroblast-like synoviocytes leading to fibroblast hyperproliferation and formation of the invasive pannus tissue that produces collagenases and stromelysin. The activated macrophages, lymphocytes, and fibroblasts also stimulate angiogenesis, which causes hypervascularization in the synovium of arthritic joints. Endothelial cells of the synovial tissue, which normally are passive and unactivated, are activated under the influence of these cytokines and start expressing variety of adhesion molecules. This activation of endothelial cells in turn helps recruitement of inflammatory cells.

Osteoclasts are derived from phagocytic precursors of the monocyte/macrophage lineage and are responsible for degradation of bone matrix. Normal skeletal remodeling is tightly controlled by a balance between osteoclasts and osteoblasts, with osteoblasts both supporting and regulating osteoclast activity. However, in pathologic states, “activated cells” (e.g., infiltrating leukocytes, synovial fibroblasts) contribute other molecules that shift the balance between osteoblastic and osteoclastic activities resulting in a higher rate of osteoclast turnover and hence, greater bone resorption. Osteoclatogenesis appear to occur in three distinct stages. In the first stage, monocytes infiltrate the affected joints. In the second stage, these infiltrating monocytes differentiate into TRAP-positive mononuclear cells, which are then induced and maintained by nurse-like cells from synovial tissues of patients with RA (RA-NLCs). In the third and final stage, cytokine-induced differentiation of these mononuclear cells into osteoclasts occurs.

Collagen-induced arthritis (CIA) in mice is the most widely used model for rheumatoid arthritis. This animal model was described by Trentham et al. (1977, J. Exp. Med., 146:857-68). The histopathology of CIA is characterized by a proliferative synovitis and pannus formation that erodes the adjacent cartilage and ultimately produces severe articular injury and ankylosis. CIA shares clinical, histological, and immunological features with human RA and has therefore been regarded as an important model of this human disease. This model is reproducible, well defined, and has proven useful for development of new therapies for rheumatoid arthritis. Arthritis-like symptoms and signs (i.e., CIA) can be induced in susceptible mouse strains following an intradermal immunization with collagen-II (CII) emulsified in an adjuvant.

Poxvirus-Encoded Complement Inhibiting Proteins

The vaccinia virus major secretory protein, referred to as the vaccinia virus complement control protein (VCP), is a Poxvirus-encoded complement inhibiting protein. VCP contains four short consensus repeats that are most similar in sequence (38% identity) to the first four repeats of C4b binding protein, one of the inhibitors of classical complement pathway. Infected cells secrete VCP after cleavage of signal peptide and can inhibit the classical as well as alternate complement pathway. VCP blocks the complement pathway by binding to the third and fourth complement components and by blocking the formation of the C3-convertase as well as by accelerating the decay of the convertase. In addition, VCP has been shown to cleave C3b in the presence of factor I to iC3b. VCP is a 35 kD protein that is made up of 243 amino acid residues.

Structurally, VCP consists of four short consensus repeats (SCRs) which bear 38, 35, 31% amino acid identity to C4b-BP, MCP, and DAF, respectively. Apart from complement regulation, VCP shares another common property with other complement regulators, for example, MCP, DAF and CR1. This common property is cell-surface association, although the mechanism of surface association is different. VCP has a strong heparin binding ability. It has two heparin binding sites on modules 1 and 4 respectively. As stated before, VCP is an extended molecule. Involvement of regions of module 1 in heparin binding and complement inhibition in VCP indicates that heparin binding function of module 4 is probably more important in cell-surface association.

VCP functionally is more similar to CR1, but it appears to be more robust than CR1. Since VCP is much smaller than CR1, it will be more efficient in reaching inflamed synovium and periarticular spaces of arthritic joints. VCP can block local inflammatory reaction and tissue destruction in the joints, by blocking the activation of both alternative as well as classical complement system. VCP should inhibit anaphylatoxic effects resulting from complement activation, which includes a multitude of activities: vasopermeation and vasodilation; chemotaxis of several cell types—mast cells, neutrophils and macrophages; degranulation of basophils and mast cells; stimulation of respiratory burst by several cell types; and induction of inflammatory cytokines.

The success of the Poxvirus family of viruses is due, in no small part, to its collective ability to encode proteins that subvert the host immune systems. VCP, the major secretory protein of vaccinia virus, is one such molecule. Poxvirus-encoded complement inhibiting proteins, including VCP, are related both structurally and functionally to human C regulatory molecules. Another Poxvirus-encoded complement inhibiting protein that is a homologue of VCP in the cowpox virus has been termed “inflammation modulatory protein” (IMP) (Miller et al., 1997, Virology, 229:126-133). IMP has been shown in a mouse air-pouch model to significantly reduce the influx of inflammatory cells and drastically diminish the tissue damage elicited by cowpox virus infection (Kotwal et al., 1998, Mol. Cell. Bioclhem., 185:39-46). A VCP homologue also has been identified in monkeypox virus (Schelkunov et al., 1998, Virology, 243:432-460), variola virus, and smallpox. Various Poxvirus-encoded complement inhibiting proteins are functionally identical and, therefore, can be interchanged.

Nucleic Acids, Polypeptides, and Related Methods

The following terms are used to describe the sequence relationships between two or more nucleic acids or polypeptides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a sequence, wherein the sequence in the comparison window may include additions or deletions (i.e., gaps) compared to the reference sequence (which does not include additions or deletions) for optimal alignment of the two sequences. Generally, the comparison window is at least 10 contiguous nucleotides or amino acids in length, and optionally can be 20, 30, 40, 50, 60, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Examples of such mathematical algorithms are the algorithm of Myers and Miller (1988, CABIOS, 4:11); the local homology algorithm of Smith et al. (1981, Adv. Appl. Math., 2:482); the homology alignment algorithm of Needleman and Wunsch (1970, J. Mol. Biol., 48:443); the search-for-similarity-method of Pearson and Lipman (1988, PNAS USA, 85:2444); the algorithm of Karlin and Altschul (1990, PNAS USA, 87:2264), modified as in Karlin and Altschul (1993, PNAS USA, 90:5873).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988, Gene, 73:237), Higgins et al. (1989, CABIOS, 5:151), Corpet et al. (1988, Nucl. Acids Res., 16:10881), Huang et al. (1992, CABIOS, 8:155), and Pearson et al. (1994, Meth. Mol. Biol., 24:307). The ALIGN program is based on the algorithm of Myers and Miller, supra. The BLAST programs of Altschul et al. (1990, J. Mol. Biol., 215:403; and 1997, Nuc. Acids Res., 25:3389) are based on the algorithm of Karlin and Altschul supra.

Software for performing BLAST analyses is publicly available through the National Center for Biotechnology Information (ncbi.nlm.nih.gov on the World Wide Web). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence can be less than about 0.1, less than about 0.01, or less than about 0.001.

To obtain gapped alignments for comparison purposes, Gapped BLAST (in BLAST 2.0) can be utilized as described in Altschul et al. (1997, Nuc. Acids Res., 25:3389). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See the World Wide Web at ncbi.nlm.nih.gov. Alignment may also be performed manually by inspection.

For purposes of the present invention, comparison of nucleotide sequences for determination of percent sequence identity to the promoter sequences disclosed herein can be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the alternative program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percent sequence identity” means the value determined by comparing two optimally aligned sequences over a comparison window, wherein the portion of the polynucleotide sequence in the comparison window may include additions or deletions (i.e., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide includes a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide includes a sequence with at least 70%, 71%, 72%, 73%, 74%, 75%, 76%, 77%, 78%, 79%, 80%, 81%, 82%, 83%, 84%, 85%, 86%, 87%, 88%, 89%, at least 90%, 91%, 92%, 93%, or 94%, or 95%, 96%, 97%, 98% or 99%, sequence identity to the reference sequence over a specified comparison window. Optimal alignment may be conducted using the homology alignment algorithm of Needleman and Wunsch (1970, supra). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl (1984, Anal. Biochem., 138:267); Tₘ 81.5° C.+16.6 (log M)+0.41 (% GC)−0.61 (% form)−500/L, where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration can be increased so that a higher temperature can be used. An extensive guide to the hybridization of nucleic acids is found in Tijssen (1993, Laboratory Techniques in Biochemistry and Molecular Biology Hybridization with Nucleic Acid Probes, Part 1, Chapter 2, “Overview of principles of 15 hybridization and the strategy of nucleic acid probe assays,” Elsevier, N.Y.). Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, or about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Thus, the invention described herein includes methods using polypeptides that are substantially identical to VCP or to a homologue thereof.

The invention also includes proteins with substitutions of at least one amino acid residue in the polypeptide. Amino acid substitutions falling within the scope of the invention include those that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

Substitution of like amino acids can also be made on the basis of hydrophilicity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In such changes, the substitution of amino acids whose hydrophilicity values can be within ±2, within ±1, or within ±0.5.

In one embodiment of the invention, a Poxvirus-encoded complement inhibiting protein has a conservative amino acid substitution, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitutions also include groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

Exemplary substitutions include those in Table 1.

TABLE 1

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr; Ala; Leu |
| Thr | Ser; Ala |
| Trp | Tyr |
| Tyr | Trp; Phe |
| Val | Ile; Leu |

Poxvirus-encoded complement inhibiting proteins (e.g., VCP and homologues thereof), molecules that are substantially identical to such proteins, and such proteins that contain one or more substitutions, can all be assayed for functionality using methods known in the art. The ability of a protein to inhibit the complement cascade or to inhibit cellular infiltration can be determined using pharmacological models that are well known in the art. For example, see The Merck Manual, 17^{th} Ed., Merck Research Laboratories, Whitehouse Station, N.J., USA, 1999, page 1022; Kotwal et al., 1990, Science, 250:827; and Rosengard et al., 2002, PNAS USA, 99:8808-13. Specifically, the functionality of a Poxvirus-encoded complement inhibiting protein can be confirmed by its ability to inhibit complement-mediated lysis of sheep erythrocytes in a hemolysis microassay (Kotwal et al., 1990, supra). Such proteins also can evaluated for their effectiveness in treating rheumatic diseases.

Formulations and Administration

Salts of carboxyl groups of a Poxvirus-encoded complement inhibiting protein may be prepared in the usual manner by contacting the polypeptide with one or more equivalents of a desired base such as, for example, a metallic hydroxide base, e.g. sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of a Poxvirus-encoded complement inhibiting protein may be prepared by utilizing an N-acyl protected amino acid for the final condensation, or by acylating a protected or unprotected polypeptide. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like.

Formyl-methionine, pyroglutamine and trimethyl-alanine may be substituted at the N-terminal residue of the polypeptide. Other amino-terminal modifications include aminooxypentane modifications.

Acid addition salts of the polypeptide or of amino residues of the polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

A Poxvirus-encoded complement inhibiting protein, including its salts, can be administered to a patient. Administration of a Poxvirus-encoded complement inhibiting protein in accordance with the present invention may be in a single dose, in multiple doses, and/or in a continuous or intermittent manner, depending, for example, upon the recipient's physiological condition, whether the purpose of the administration is therapeutic or prophylactic, and other factors known to skilled practitioners. The administration of a Poxvirus-encoded complement inhibiting protein may be essentially continuous over a pre-selected period of time or may be in a series of spaced doses. The amount administered will vary depending on various factors including, but not limited to, the condition to be treated and the weight, physical condition, health, and age of the patient. Such factors can be determined by employing animal models or other test systems that are available in the art.

To prepare a Poxvirus-encoded complement inhibiting protein, the desired protein is synthesized or otherwise obtained and purified as necessary or desired. A Poxvirus-encoded complement inhibiting protein can be adjusted to the appropriate concentration, and optionally combined with other agents. The absolute weight of a Poxvirus-encoded complement inhibiting protein included in a unit dose can vary.

One or more suitable unit dosage forms including a Poxvirus-encoded complement inhibiting protein can be administered by a variety of routes including topical, oral, parenteral (including subcutaneous, intravenous, intramuscular and intraperitoneal), rectal, dermal, transdermal, intrathoracic, intrapulmonary and intranasal (respiratory) routes. A Poxvirus-encoded complement inhibiting protein can also be administered directly into a patient's joint area.

The formulations may, where appropriate, be conveniently presented in discrete unit dosage forms, and may be prepared by any of the methods well known to the pharmaceutical arts. Such methods may include the step of mixing a Poxvirus-encoded complement inhibiting protein with liquid carriers, solid matrices, semi-solid carriers, finely divided solid carriers or combinations thereof, and then, if necessary, introducing or shaping the product into the desired delivery system. By “pharmaceutically acceptable” it is meant a carrier, diluent, excipient, and/or salt that is compatible with the other ingredients of the formulation, and not deleterious or unsuitably harmful to the recipient thereof. The therapeutic compounds may also be formulated for sustained release (for example, using microencapsulation, see WO 94/ 07529, and U.S. Pat. No. 4,962,091).

A Poxvirus-encoded complement inhibiting protein may be formulated for parenteral administration (e.g., by injection, for example, bolus injection or continuous infusion) and may be presented in unit dose form in ampoules, pre-filled syringes, small volume infusion containers, or in multi-dose containers. Preservatives can be added to help maintain the shelve life of the dosage form. A Poxvirus-encoded complement inhibiting protein and other ingredients may form suspensions, solutions, or emulsions in oily or aqueous vehicles, and may contain formulatory agents such as suspending, stabilizing and/or dispersing agents. Alternatively, a Poxvirus-encoded complement inhibiting protein and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers and vehicles that are available in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold inder the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils, and polysiloxanes.

It is possible to add other ingredients such as antioxidants, surfactants, preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions at a pH of about 7.0-8.0.

Furthermore, a Poxvirus-encoded complement inhibiting protein may also be used in combination with other therapeutic agents, for example, pain relievers, anti-inflammatory agents, antihistamines, and the like, whether for the conditions described or some other condition.

The present invention further pertains to a packaged pharmaceutical composition such as a kit or other container. The kit or container holds a therapeutically effective amount of a pharmaceutical composition for and instructions for using the pharmaceutical composition for treating a condition.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the invention described in the claims.

### EXAMPLES

### Example 1

### VCP Treatment of Rheumatic Disease

Arthritis was induced in male DBA/1J mice by injecting 200 μg of bovine type II collagen in complete Freund's adjuvant by intradermal injection at the base of the tail. Arthritic mice were treated with intraperetoneal (i.p.) injections of VCP according to one of the following treatment schedules: VCP injection (25 mg/kg of animal weight) two times a day starting at disease onset (days 9-16), early intervention (days 16-23) and delayed intervention (days 24-31). Inflammation was monitored by already established arthritic scoring, and also by measuring hind paw thickness. Morphologic appraisals of inflammation, bone damage, and integrity of the articular cartilage matrix were studied by histological sections of arthritic hind paws. The results show that VCP blocks inflammation in all three different treatment schedules. In addition to lowering the clinical arthritic scores and inflammation in arthritic mice, VCP also prevented loss of cartilage matrix proteoglycans. Lesser numbers of infiltrating inflammatory cells were detected in VCP treated arthritic joints. Immunohistohemical studies of the arthritic joints show that the number of osteoclasts is reduced in VCP treated arthritic mice. The information presented herein indicates that VCP not only blocks inflammation but also inhibits bone erosion.

### Example 2

### SDS/Polyacrylamide Gel Electrophoresis

NuPage® Novex pre-cast Bis-Tris gels were used with a Novex gel box. Then 200 ml running buffer with 500 μl antioxidant was added inside with the gel, and 800 ml running buffer without antioxidant was added outside of the gel in the gel box. The comb was taken out, and the samples were loaded. The samples were prepared as follows: 10 μl sample, 2 μl reducing agent, 5 μl loading buffer and 3 μl H₂O. The mixture was incubated at 70° C. for 10 min before loading. For the rainbow marker: 4 μl marker, 5 μl loading buffer, 2 μl reducing agent and 9 μl H₂O was mixed and incubated at 100° C. for 3 minutes before loading. The gel was run under 195 voltages for about 30 minutes.

### Example 3

### Hemolysis Assay

3.0 ml sensitized sheep RBCs (Red blood cells) were centrifuged briefly at 7000 rpm. Then 1.5 ml supernatant was taken out and saved as reaction buffer. The remaining solution was mixed by vortexing. Then the serum was prepared at a 1:70 (V/V) ratio in reaction buffer. Microfuge tubes were set on ice when the reaction mixture was prepared. For each reaction, 150 μl RBC solution, 50 μl reaction buffer, 20 μl sample (cell medium collected and concentrated), and 30 μl serum were added in sequence. For the negative control, no serum was added, but cell medium from the negative control of transfection was added. For the positive control, rVCP DNA was used to replace the samples. Then the reactions were incubated at 37° C. for exactly one hour and centrifuged at 10,000 rpm for 30 seconds. 150 μl of the supernatant was taken out and added to each well of the plate as follows: negative control, positive control, sample one, sample two, . . . etc. Absorbance was checked at 405 nm for calculation of percentage of inhibition of the lysis of sheep RBCs, an indication of VCP activity.

### Example 4

### Western Blot Analysis

Novex Western transfer apparatus and Hybond ECL nitrocellulose membrane were used for Western blot analysis. A gel membrane sandwich in the transfer apparatus was made for transferring proteins from SDS gel to nitrocellulose membrane. From the cathode core (−) to the anode core (+), the components of the sandwich were: blotting pad, blotting pad, filter paper, gel, nitrocellulose membrane, filter paper, blotting pad, blotting pad. The sandwich was also assembled by sequence from cathode (−) to anode (+). The blotting pads were soaked in transfer buffer for 60 minutes, and all the air bubbles in them were removed before transfer. Nitrocellulose membrane and filter paper were also soaked in transfer buffer for several minutes before transfer. The blot module was then inserted into the buffer chamber, and transfer buffer was added so that the gel membrane sandwich was totally covered by the buffer. It was run at 35 mA for 2 hours before immunodetection. The membrane was then blocked in 5% (w/v) blocking reagent in PBS-T (Tween-20 concentration in PBS is 0.12%) for one hour at room temperature on a shaker. After non-specific binding sites were blocked, the membrane was rinsed twice briefly, and then washed once for 15 minutes, twice for 5 minutes in PBS-T. Then the membrane was incubated in diluted primary rat antibody for VCP (1:2000, v/v) for 1 hour at room temperature. The membrane was washed the same as after blocking, and then was incubated with the peroxidase labeled anti-rat secondary antibody (1:2000, v/v) for 1 hour at room temperature. The membrane was washed the same as after blocking. Excess wash buffer was drained from the membrane, and ECL detection reagent (mixed by equal volume of detection solution 1 and detection solution 2) was added to cover the membrane. After 1-minute incubation, excess detection reagent was drained off the membrane, and the membrane, wrapping up in a sheet of Saran Wrap, was exposed to the film as soon as possible.

### Example 5

### Serum Collection

The blood was collected in micro-centrifuge tubes by cutting the tip of the tail. The blood was allowed to clot at room temperature for 1 hour and then centrifuged at 7,000 rpm (Sorvall centrifuge H-1000B) for 5 minutes. The serum was then recovered and frozen until antibody titer determination.

### Example 6

### Immunohistochemistry Studies

The immunohistochemistry was done as follows using paraffin embedded hearts. After the tissue was mounted, the slides were deparaffinized and hydrated by soaling the slides for 2 minutes each in xylene, 100% ethanol, 95% ethanol, 70% ethanol, and 50% ethanol. The slides were placed in 3% hydrogen peroxide for 5 minutes to quench endogenous peroxidase. They were rinsed and incubated for 10 minutes with Protease to unmask the antigenic markers for IgG and IgM and EDTA to unmask the antigenic markers for VCP. Again, the slides were rinsed and incubated for 20 minutes with serum from the same species as the origin of the secondary antibody. After rinsing, they were incubated for 60 minutes with one of the following antibodies: goat anti-rat IgM (Pierce), goat anti-rat IgG (Pierce), or chicken IgY anti-VCP (Washington Biotec). The slides were again rinsed and incubated for 60 minutes with one of the following secondary antibodies, biotinylated rabbit anti-rat IgG (Vector Laboratories), biotinylated goat anti-chicken IgG (Vector Laboratories). After rinsing the slides were incubated for 30 minutes with Vectastain elite ABC reagent (Vector Laboratories). A final rinsing was followed by incubation with Diaminobenzidine tablets (Sigma) for 5 minutes. Next, the slides were counter stained with diluted H & E (2 minutes for each stain) followed by dehydration (the reverse of the hydration step) and mounted with Permount (Fisher Scientific).

### Example 7

### Induction of Collagen-Induced Arthritis

Bovine type II collagen (BCII) was solubilized to a concentration of 2 mg/ml in 0.01 M acetic acid at 4° C. with constant mixing overnight. For induction of CIA, BCII was emulsified with an equal volume (1:1) of CFA (Difco, Detroit, Mich.). Male DBA/1 mice at 7-9 wk of age received 200 μg of bovine type II collagen (CII; Chondrex) in CFA (Chondrex) by intradermal injection (day 0). Mice were monitored daily for signs of arthritis for which severity scores were derived as described below.

### Example 8

### Clinical Evaluation of CIA

Arthritis development was assessed by inspection three times a week by a blinded examiner. Clinical severity of arthritis was quantified according to a graded scale from 0 to 3 as follows: 0, normal; 1, detectable swelling in one joint; 2, swelling in more than one but not in all joints; and 3, severe swelling of the entire paw and/or ankylosis. Each paw was graded, and each mouse could achieve a maximum score of 12. A mean arthritic score value was calculated.

### Example 9

### Efficacy of VCP to Suppress CIA

Mice were given VCP (25 mg/kg/day) according to 1 of the following 3 schedules: daily VCP beginning at the onset of arthritis (days 9-33), a regimen that has been shown to be effective against AIA, an early intervention protocol (days 15-33), or a delayed intervention protocol (days 21-33). All animals were necropsied 3 day after the end of treatment.

### Example 10

### Histopathology

For histological assessment, mice were sacrificed and the hind limbs removed and fixed in 10% neutral-buffered formalin, then decalcified in 5% formic acid and embedded in paraffin. Sections (5 μm) were stained with H&E or toluidine blue (Sigma-Aldrich).

### Example 11

### TRAP Staining

Tissues were fixed in 10% neutral buffered formalin, decalcified, washed in water, processed and embedded in paraffin. Tissues were sectioned at 4 microns, floated on a protein free water bath, picked up on charged glass slides (Thermo Shandon), air dried overnight, dried in a 58° C. oven overnight. Slides were dewaxed, hydrated to distilled water, placed in Target Retrieval Solution (DakoCytomation), and placed in a 75° C. oven overnight for antigen retrieval. Because the tissues were becoming slightly detached from the slides, manual staining was performed. Slides were incubated for 5 minutes in 3% hydrogen peroxide to block endogenous peroxidase activity, and washed in distilled water followed by tris buffer, 2×2 minutes each. The Vector MOM kit was used for the detection system (Burlingham, Calif.). Slides were incubated for 1 hour in a working solution of MOM (Mouse Ig Blocking Reagent), [2 drops of MOM to 2.5 ml of Tris buffer]. Slides were washed in Tris buffer for 2×2 minutes each. Slides were incubated for 5 minutes in a working solution of MOM diluent [600 ml of protein concentrate stock solution to 7.5 ml of Tris buffer]. Excess diluent was drained off and the primary antibodies were added for a 1-hour incubation. Primary antibody (clone 9C5, 1:1000 dilution, from Anthony Janckila, VAMC, Louisville, Ky., available from Zymed Laboratories) was used, as was clone 26E5, 1:50, NCL-TRAP from Novocastra (Vector Laboratories). Diluting buffer S0809 from DakoCytomation was used in place of a primary antibody on the negatives. Slides were washed in Tris buffer for 2×2 minutes each. All slides were incubated for 10 minutes in MOM biotinylated anti-mouse IgG Reagent prepared as follows: 10 μl of stock solution to 2.5 ml of MOM diluent prepared as above. Slides were washed in Tris buffer for 2×2 minutes each. Slides were incubated for 5 minutes in Vectastain Elite ABC reagent prepared as follows: add 2 drops of Reagent A to 2.5 ml of Tris buffer, add 2 drops of Reagent B to the 2.5 ml of Tris, mix well, and allow to stand for 30 minutes prior to use. Slides were washed in Tris buffer for 2×2 minutes each. Slides were incubated in DAB (DakoCytomation), for 10 minutes. Slides were washed in distilled water. Slides were incubated in Aqueous Hematoxylin (Biomeda) for 1 minute. Slides were washed in distilled water. Nuclei were ‘blued’ in automation buffer (Biomeda) for 1 minute. Slides were washed in distilled water. Slides were dehydrated, cleared in xylenes, and coverslipped using Permount.

### Example 12

### Detection of Serum Antibodies to C-II

Serum antibody titers were measured by an ELISA assay. In brief, a 96-well microplate (Falcon®, Becton Dickinson Labware, Franklin Lakes, N.J.) was coated with 50 μl/well of a 20 μg/ml solution of bovine C-II or mouse C-II (Elastin Products Company, Inc., Owensville, Mo.) in PBS at 4° C. overnight, washed three times with PBS containing 0.05% Tween 20 and 0.1% BSA, and then blocked with 250 μl/well of PBS containing 0.2% BSA at 4° C. overnight. The diluted serum (1/400-1/20,000) was added at 50 μl/well and allowed to react at 4° C. overnight. The wells were washed three times with PBS containing 0.05% Tween 20, incubated with 50 μl of a 1:200 dilution of goat anti-mouse IgG1, IgG2a, IgG2b, or IgM coupled with horseradish peroxidase (Binding Site Inc., San Diego, Calif.) at 4° C. for 2 h, washed three times with PBS containing 0.05% Tween 20, and developed at room temperature for 30 min with 0.1 ml of TrueBlue Peroxidase Substrate (Kirkegaard and Perry Labs, Gaithersburg, Md.). The OD at 450 nm was read using a microplate reader (Bethyl Inc., Montogomery, Tex.).

### Example 13

### ELISA

All cytokines levels were detected by ELISA. TNF-α, IL-6, and IL-10 (all BD PharMingen, San Diego, Calif.) assays were performed according to the manufacturer's instructions. Detection limits were as follows: IL-6, and TNF-α all at 10 pg/ml; IL-12 at 20 pg/ml.

### Example 14

### Knee and Paws Radiology

Upon sacrifice, knees and paws were promptly removed to ice, and x-rays were taken. The samples were positioned over a radiographic cassette (Detector: Trophy RVG-ui, Marne-la-Vallée, France) to obtain a lateral view. A conventional x-ray source GE 1000 AC generator (Milwaukee, Wis.) was used at exposure factors of 55 kV (peak) and 15 mA with source-to-object distance of 54 cm.

### Example 15

### Establishment of Collagen-Induced Arthritis

The CIA model is a commonly used mouse model of human rheumatoid arthritis in which disease is induced by immunization of DBA/1 mice with native CII in CFA in the dermis near the base of tail. In this model, immunization of mice bearing a permissive H2 haplotype with heterologous bovine CII and CFA initiates a T-dependentB cell response to autologous (mouse) CII. Extensive necrosis of the tail at the injection site was visible 7-21 days postimmunization. This necrosis is eventually resolved in all groups, although scarring remains as evidenced by kinks in the tails at the injection site. The first clinical sign of arthritis appears approximately at day 21 post-immunization. Usually disease affects only one to three paws at a given time point. Typically, clinical signs include swelling of one or more digits, severe edema and ankylosis. The incidence of arthritis was 100%, although severity of clinical signs varied in a given group at a given time point.

### Example 16

### VCP is Delivered to Arthritic Joints When Given I.P.

Synovial tissue is the major site in the pathogenesis of RA as well as CIA. To study the presence of VCP in the synovial tissue, arthritic mice were injected with 25 mg/kg VCP on day 30 (post immunization with collagen) via i.p. The mice were sacrificed and the right knee was dissected. The knee was fixed and embedded in paraffin. 5 μm thick sections were immunostained for VCP using rabbit-anti-VCP. VCP was observed in the synovial tissue and at the site of pannus formation.

### Example 17

### VCP Suppresses the Inflammation in Collagen-Induced Arthritis

The anti-inflammatory effects of VCP on type-II collagen-induced arthritis in DBA1/J mice were examined. This animal model is widely used for evaluation of antirheumatic drugs because of its pathological similarities to human rheumatoid arthritis.

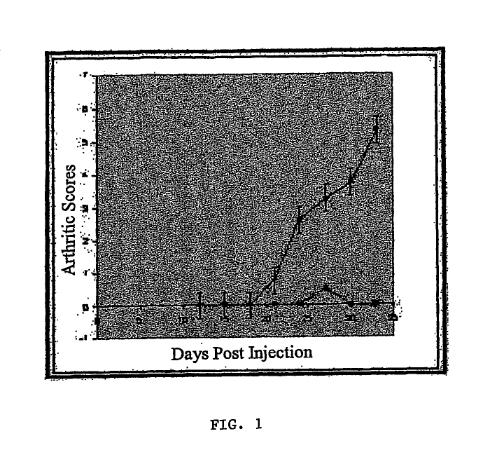

The animals were randomly divided into three groups (n=5 in each group). The arthritis was elicited in two groups. To study the effect of VCP on CIA, one of the two groups with arthritis received 25 mg/kg of animal weight VCP (i.p.) twice every day from day 9 onwards till the end of the experiment (day 33). Animals treated with VCP were compared with those that were administered PBS as the vehicle control. As shown in FIG. 1, VCP markedly inhibited type-II-collagen-induced arthritis in mice when administered at early stage of the disease. No sign of inflammation or edema was observed in VCP treated animals.

### Example 18

### VCP Reduces the Disease Severity After the Disease Onset and in Established Disease

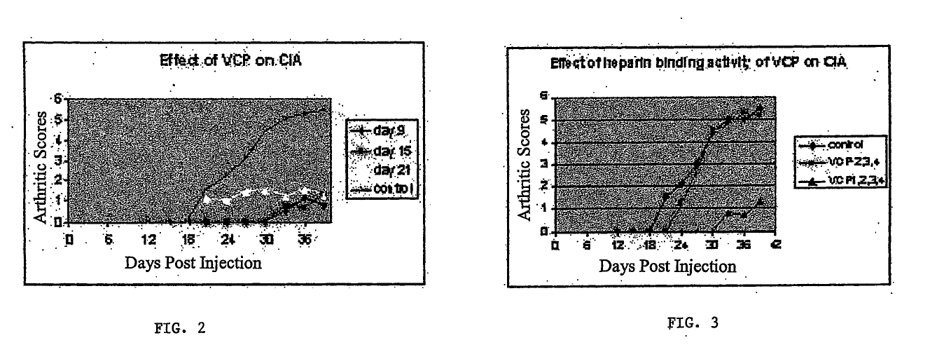

To investigate whether the treatment with VCP is still effective after the onset of disease, a delayed treatment was performed. Treatment was started from day 15 and the clinical scores were examined. Arthritis was induced in DBA-1/j mice and divided randomly in two groups. One group received 25 mg/kg of VCP twice daily via i.p. from day 15 to day 33 (delayed treatment). The delayed treatment was also effective in reducing the clinical arthritis scores (FIG. 2). These results demonstrated that the treatment with VCP either before or after the onset of disease could inhibit the disease severity of CIA, indicating the involvement of the complement pathway in the early stages of pathogenesis of CIA.

To study the effect of VCP on established form of disease, animals showing signs of disease in one or more paws on day 21 were divided into two groups (five animals per group). From days 21-33, animals in one group received twice daily an i.p. dose of VCP (25 mg/kg), whereas those in the other group received an equal volume of i.p. saline. The differences in the clinical scores are shown in FIG. 2. Intraperitoneal administration of VCP had a drastic inhibitory effect on the progression of disease as assessed by clinical score. The difference in clinical score between treated and untreated animals was maintained throughout the course of the experiment (FIG. 2).

### Example 19

### Contribution of Heparin Binding Activity on Therapeutic Effects of VCP

In addition to binding to complement components, VCP also binds to heparin. Heparin is present on surface of variety of cells. VCP binds to the endothelial cells. To study the effect of heparin binding activity of VCP on CIA, truncated VCP (VCP-2,3,4), which lacks domain that binds to complement component, was used. This truncated form of VCP retains its ability to bind heparin.

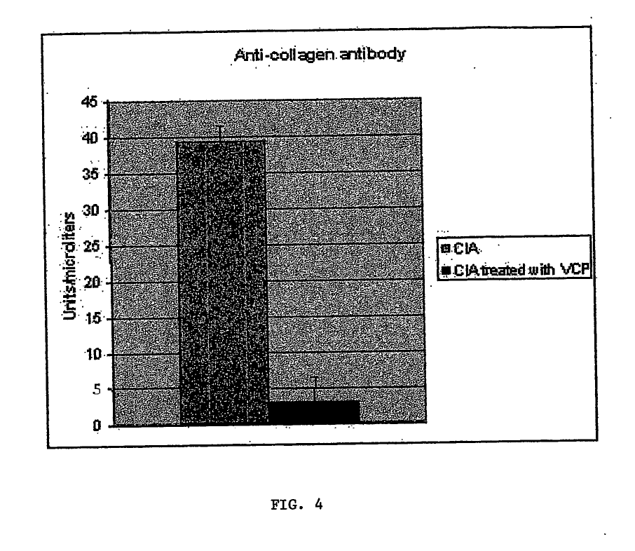

Two groups of mice were injected with CII-CFA and one group received VCP-2,3,4 (25 mg/kg of body weight) and the other group received PBS. No reduction in disease progression was observed in treated group. A slight delay in appearance of first clinical sign was observed in VCP-2,3,4 treated group but apart from that there was no difference in progression and clinical severity in the two groups (FIG. 3). This result indicates that heparin-binding activity of VCP alone does not have any therapeutic effect on CIA. Heparin binding ability alone did not help in any inhibition in cartilage loss and pannus formation as revealed by Toluidine blue staining.

### Example 20

### Production of Anti-Collagen Abs

To examine whether treatment with VCP affected humoral immunity to collagen, serum levels of anticollagen Ab were measured on days 21 and 33. Anticollagen Abs were not detected in the sera of nonimmunized DBA control mice; the low levels of Ab present on day 21 of the immunized mice were not different among the three treatment groups. Increased serum levels of anticollagen Ab were observed between days 21 and33 in the immunized mice treated with PBS alone. However, the changes in serum level of anticollagen Ab over 3 weeks were markedly reduced after treatment with VCP (FIG. 4).

### Example 21

### Down-Regulation of Pro-Inflammatory Cytokines in the Joints by VCP Treatment

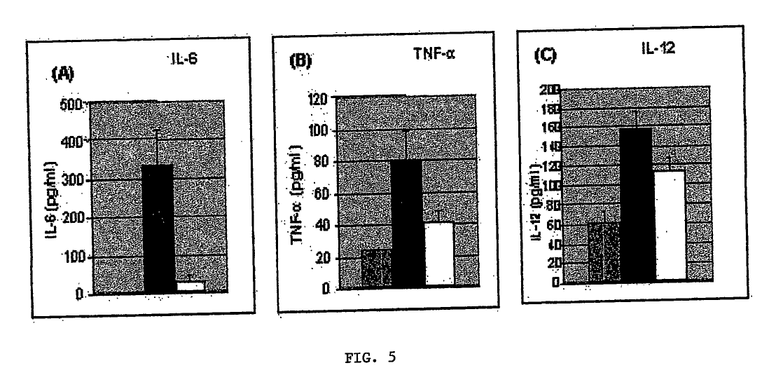

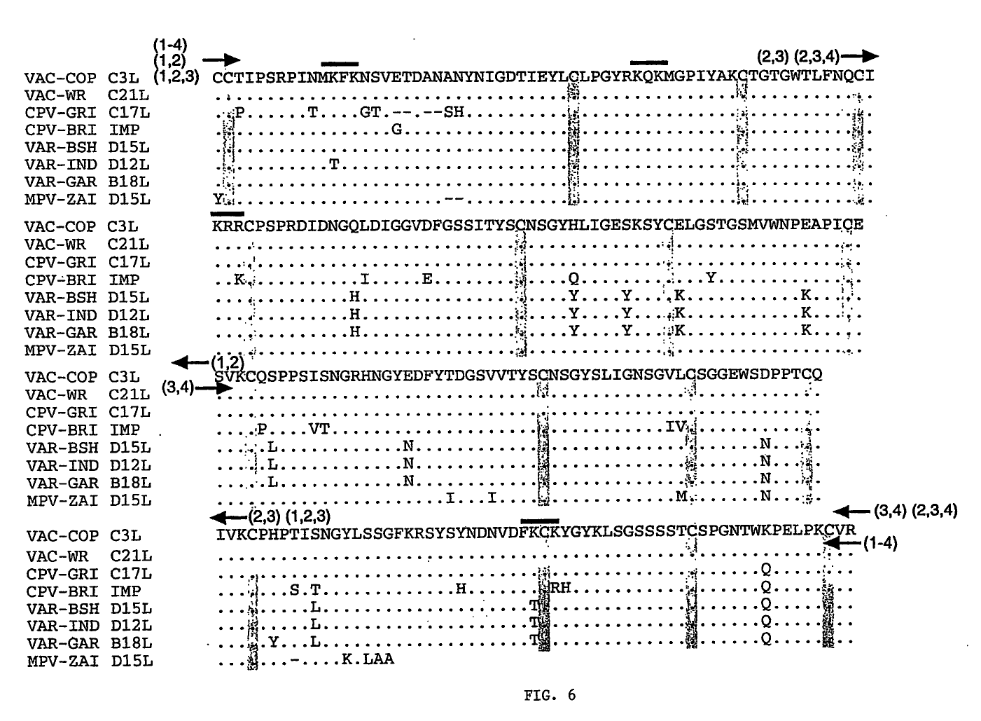

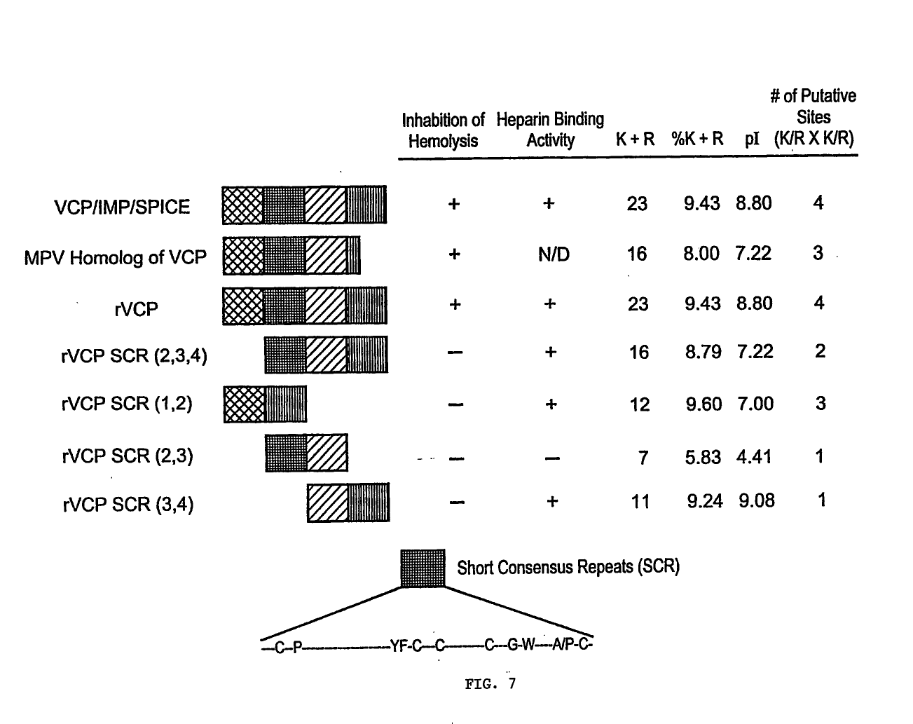

Since the inflammatory process in the synovium plays a major role in the development of arthritis), the change of proinflammatory cytokine expression in the arthritic mice by the treatment was examined. High levels of TNF-α and IL-6 were observed in the serum from the control untreated arthritic mice. The treatment with VCP significantly inhibited the expression of these proinflammatory cytokines (FIGS. 5A and 5B). These results indicated that the VCP treatment reduced the arthritic manifestations by down-regulating the expression of proinflammatory cytokines in the joints.

### Example 22

### rVCP Treatment Reduces the Level of IL-12

IL-12 is a heterodimeric cytokine that has many immunoregulatory properties. It consists of a p40 and a p35 subunit and is released by antigen-presenting cells like monocytes/macrophages in response to bacterial products and immune signals. In addition to enhancing natural killer (NK)-mediated cytotoxicity it also plays a key role in promoting Th1 immune responses. The level of IL-12 in sera of VCP-treated and untreated arthritic mice was determined. Reduction in IL-12 was observed in serum samples from VCP treated group as compared to the untreated arthritic group (FIG. 5C).

### Example 23

### Histopathologic Changes

To assess histopathologic changes in mice, five animals in each group were euthanized at day 33 after induction of CIA, and hematoxylin and eosin stained sections of the knee joints were examined. Representative tissue sections of joints from normal, untreated control, and VCP treated mice were examined. Normal mice without CIA induction showed intact cartilage with a slight protrusion of the synovium into the joint space at the joint margin. No signs of pannus formation could be seen. Untreated control CIA groups showed marked inflammation of the synovium and sub-synovial tissue with pannus formation eroding through cartilage and deep into subchondral bone. Cartilage destruction was diffuse and severe. The knee joints and hind paws of mice with CIA revealed massive neutrophilic and mononuclear cell infiltration, loss of articular cartilage, and bone erosions typical of advanced CIA. Therefore, treated animals not only had decreased clinical symptoms of disease, but the bone, synovium, and cartilage of the joint were preserved. In comparison to the control untreated, the severity of histopathologic changes was markedly reduced in mice given VCP. Joint space narrowing and necrosis are evident in untreated groups. In contrast, joints from animals treated with VCP were relatively normal in appearance, with well-preserved joint architecture and joint space. In particular, there was amelioration of synovial and sub-synovial inflammation, such that pannus formation and cartilage destruction was minimal and restricted to the joint margins. The treatment with VCP resulted in reduced neutrophilic and mononuclear cell infiltration to the joint space.

### Example 24

### VCP Inhibits the Cartilage Destruction in Arthritic Joints

One of the hallmarks of RA is the cartilage destruction mediated by infiltrating inflammatory cells and cytokines released by these cells. The knee joints from normal and both VCP-treated and untreated mice were dissected and embedded in paraffin. Sections were then stained with toluidine blue which stains the proteoglycans of the cartilage in the joints. After toluidine blue staining, the cartilage was seen as dark blue to purple color, while the rest of the tissue was stained in different shades of light blue. VCP-treated joints retained their cartilage integrity, whereas in untreated arthritic knees, drastic loss of cartilage could be seen by a loss of toluidine blue stain.

### Example 25

### VCP Treatment Prevents Bone Erosion and Preserves Joint Integrity

Whole knee joints were radiologically studied for the degree of joint destruction. X-ray photographs of PBS- and VCP-treated arthritic mice were taken at days 31 and 38 after immunization with collagen-II. In knee joints of controls from the PBS-treated group and untreated groups, marked zones of bone erosions were found in the femur the patella. Joint space was markedly reduced and signs of increased bone resorption were observed. Clear protection against joint destruction after VCP treatment in established CIA was observed. Marked prevention of joint damage was still noted in the VCP treated group compared with the control group on day 38. The joint space was found to be intact and comparable to the normal non-arthritic joints.

### Example 26

### VCP Reduces the Osteoclast-Like Cells in Joints

Osteoclasts play a key role in bone resorption. To examine potential inhibition of osteoclastic activity after VCP administration, TRAP staining was performed on paraffin-embedded knee joint sections. TRAP activity is a characteristic phenotypic marker of osteoclasts and osteoclast precursors and is expressed in osteoclast-like cells in mice with well-established collagen arthritis. Mice of the control PBS treated group revealed bone erosion and high numbers of TRAP⁺ osteoclast-like cells were seen in mice that had developed marked arthritis. However, all mice of the VCP treated group showed marked reduction in TRAP⁺ osteoclast-like cells at sites of erosion in subchondral, trabecular, and cortical bone of the patella and femur/tibia region.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a patient having a rheumatic disease or at risk of developing a rheumatic disease, comprising administering to the patient an effective amount of Poxvirus-encoded complement inhibiting protein and a pharmaceutically-acceptable carrier, wherein the effective amount of the Poxvirus-encoded complement inhibiting protein treats at least one symptom associated with the rheumatic disease.

2. The method of claim 1, wherein the rheumatic disease is selected from the group consisting of osteoarthritis, rheumatoid arthritis, juvenile rheumatoid arthritis, fibromyalgia, systemic lupus, erythematosus, scleroderma, a spondyloarthropathy, ankylosing spondylitis, reiter's syndrome, gout, infectious arthritis, lyme disease, polymyalgia rheumatica, polymyositis, psoriatic arthritis, bursitis, and tendinitis.

3. The method of claim 1, wherein the rheumatic disease is arthritis.

4. The method of claim 1, wherein the rheumatic disease is rheumatic arthritis.

5. The method of claim 1, wherein administration of the Poxvirus-encoded complement inhibiting protein suppresses inflammation.

6. The method of claim 5, wherein the administration of the Poxvirus-encoded complement inhibiting protein suppresses joint inflammation.

7. The method of claim 6, wherein the joint is a knee, wrist, finger, neck, shoulder, elbow, hip, ankle or foot joint.

8. The method of claim 1, wherein the administration of the Poxvirus-encoded complement inhibiting protein reduces cartilage damage.

9. The method of claim 8, wherein the administration of the Poxvirus-encoded complement inhibiting protein reduces cartilage damage in a joint.

10. The method of claim 9, wherein the joint is a knee, wrist, finger, neck, shoulder, elbow, hip, ankle or foot joint.

11. The method of claim 1, wherein administration of the Poxvirus-encoded complement inhibiting protein reduces bone damage or loss.

12. The method of claim 11, wherein administration of the Poxvirus-encoded complement inhibiting protein reduces bone damage or loss in a joint.

13. The method of claim 12, wherein the joint is a knee, wrist, finger, neck, shoulder, elbow, hip, ankle or foot joint.

14. The method of claim 1, wherein administration of the Poxvirus-encoded complement inhibiting protein reduces rheumatoid nodules.

15. The method of claim 1, wherein administration of the Poxvirus-encoded complement inhibiting protein inhibits osteoclast formation.

16. The method of claim 1, wherein administration of the Poxvirus-encoded complement inhibiting protein delays the onset of at least one symptom of the rheumatic disease.

17. The method of claim 1, wherein administration of the Poxvirus-encoded complement inhibiting protein prevents the onset of at least one symptom of the rheumatic disease.

18. The method of claim 1, wherein the Poxvirus-encoded complement inhibiting protein is VCP.

19. The method of claim 1, wherein the Poxvirus-encoded complement inhibiting protein is IMP.

20. The method of claim 1, wherein the Poxvirus-encoded complement inhibiting protein is smallpox complement inhibiting protein.

21. The method of claim 1, wherein the Poxvirus-encoded complement inhibiting protein is monkeypox complement inhibiting protein.

22. The method of claim 1, wherein the Poxvirus-encoded complement inhibiting protein is variola virus complement inhibiting protein.

23. The method of claim 1, wherein the Poxvirus-encoded complement inhibiting protein is administered intraperitoneally.

24. The method of claim 1, wherein the Poxvirus-encoded complement inhibiting protein is administered into a joint area.

25. The method of claim 1, wherein the Poxvirus-encoded complement inhibiting protein is administered in multiple administrations.

26. The method of claim 1, wherein the Poxvirus-encoded complement inhibiting protein is administered at a dosage of at least about 25 mg/kg.

27. The method of claim 1, wherein the Poxvirus-encoded complement inhibiting protein is administered in combination with at least one additional agent.

28. The method of claim 27, wherein the additional agent is selected from the group consisting of a nonsteroidal anti-inflammatory drug (NSAID), a disease-modifying antirheumatic drug (DMARD), and a corticosteroid.

29. The method of claim 28, wherein the NSAID is an agent selected from the group consisting of plain aspirin, buffered aspirin, ibuprofen, ketoprofen, naproxen, celecoxib, and rofecoxib.

30. The method of claim 28, wherein the DMARD is an agent selected from the group consisting of an antimalarial agent, an immunosuppressant, penicillamine, sulfasalazine, and gold.

31. The method of claim 30, wherein the antimalarial agent is hydroxychloroquine.

32. The method of claim 30, wherein the immunosuppressant is methotrexate, azathioprine, cyclosporine, or lefluomide.

33. The method of claim 28, wherein the corticosteroid is prednisone or methylprednisone.

34. A method for treating a patient having a rheumatic disease or at risk of developing a rheumatic disease, comprising administering to the patient an effective amount of Poxvirus-encoded complement inhibiting protein and a pharmaceutically-acceptable carrier, wherein the Poxvirus-encoded complement inhibiting protein is selected from the group consisting of VCP, IMP, monkeypox complement inhibiting protein, smallpox complement inhibiting protein, and variola virus complement inhibiting protein, wherein the effective amount of the Poxvirus-encoded complement inhibiting protein treats at least one symptom associated with the rheumatic disease.
